(19) United States
(12) Patent Application Publication
(10) Pub. No.: US 2019/0029966 A1
(43) Pub. Date: Jan. 31, 2019

(54) ABUSE-RESISTANT PHARMACEUTICAL FORMULATIONS

(71) Applicant: INDIVIOR UK LIMITED, Slough Berkshire (GB)

(72) Inventors: Victor Morrison Young, Edinburgh (GB); Michael Forbes, Edinburgh (GB); Alyn Brandon McNaughton, Livingston (GB)

(21) Appl. No.: 16/083,364

(22) PCT Filed: Mar. 8, 2017
(86) PCT No.: PCT/IB2017/051362

§ 371 Date: Sep. 7, 2018

Related U.S. Application Data
(60) Provisional application No. 62/305,937, filed on Mar. 9, 2016.

(51) Int. Cl. A61K 9/48; A61K 47/38; A61K 47/10; A61K 45/06

(52) U.S. Cl. CPC A61K 9/4866; A61K 45/06; A61K 47/10; A61K 47/38

(57) ABSTRACT

The disclosure provides orally administrable capsules comprising abuse-resistant, pharmaceutical formulations which comprise abuse-susceptible active agents such as opioids. The formulations provide abuse resistance by having a sufficiently high viscosity that substantially prevents the formulation and opioid therein, from being drawn into a syringe, at room temperature or higher, either alone or when mixed with water or other liquids.

### CROSS-REFERENCE TO RELATED APPLICATION

This application claims priority to U.S. Application No. 62/305,937 filed Mar. 9, 2016, the disclosure of which is incorporated by reference herein in its entirety.

### FIELD

The disclosure provides orally administrable capsules comprising abuse-resistant, pharmaceutical formulations which comprise abuse-susceptible active ingredients, such as opioids. The formulations provide abuse resistance by having a sufficiently high viscosity that substantially prevents the formulation and abuse-susceptible active ingredients therein, from being drawn into a syringe, at room temperature or higher, either alone or when mixed with water or other liquids.

### BACKGROUND

Many compounds, such as opioids are sometimes the subject of abuse. Typically, a particular dose of an active ingredient is more potent when administered parenterally when compared to the same dose administered orally via the oral mucosa or the gastrointestinal tract. Therefore, one popular mode of abuse of oral formulations involves the extraction of the abuse-susceptible active ingredient from the dosage form, mixing with liquid for some dissolution of the abuse-susceptible active ingredient out of the dosage form, and then subsequently injecting the abuse-susceptible active ingredient to achieve a high. Oral dosage formulations of abuse-susceptible active ingredients can also be crushed and snorted to achieve a high.

For the treatment of opioid dependence, buprenorphine is available as SUBUTEX® tablets (buprenorphine; Indivior PLC), which are formulated for sublingual administration. SUBUTEX® tablets are soluble in aqueous media, making it possible for addicts to misuse the pharmaceutical formulation by dissolving the tablets in water, and then injecting the resulting solution. Because SUBUTEX® tablets do not contain naloxone, they do not have the abuse-resistant attributes of other pharmaceutical formulations, such as SUBOXONE® Tablets or SUBOXONE® Film (buprenorphine/naloxone; Indivior PLC). Sublingual administration of buprenorphine has about 50% bioavailability, while the oral administration of buprenorphine only has about 5% bioavailability. Thus, in addition to the possibility of misuse, the SUBUTEX® tablets must be kept under the tongue until they dissolve, which can take up to 15 minutes.

There is a need in the art for pharmaceutical formulations that are resistant to abuse, that do not need to be administered buccally or sublingually, that do not need to contain an opioid antagonist, and that have high bioavailability when swallowed. The disclosure is directed to these, as well as, other important ends.

### BRIEF DESCRIPTION OF THE FIGURE

FIG. 1. Comparison of the dissolution data utilizing three different apparatus: Basket (USP Apparatus I) (the release profile represented with triangles); Paddle (USP Apparatus II) (the release profile represented with squares); and Reciprocating (USP Apparatus III) (the release profile represented with X). All the dissolution tests described herein refer to the USP Dissolution Test, Chapter 711 (Dec. 1, 2011). FIG. 1 also provides the mean % release quantitative disintegration (the release profile with diamonds), as measured using the USP Disintegration Test, Chapter 701 (Aug. 1, 2008).

### SUMMARY

The disclosure provides abuse-resistant pharmaceutical formulations containing an abuse-susceptible active ingredient (such as an opioid) and excipients, such as viscosity enhancing agents and gelling agents. The relatively high viscosity of the pharmaceutical formulations described herein prevents extraction of the abuse-susceptible active ingredient from the pharmaceutical formulation, which prevents abuse of the abuse-susceptible active ingredient.

There are several different characteristics that may make the pharmaceutical formulations described herein abuse resistant. One characteristic is that the high viscosity substantially prevents the formulation from being drawn into a syringe, even when the formulation is mixed with water or alcohol. Another characteristic is that the viscosity increases upon heating the formulation in water or alcohol. In this embodiment, upon exposure to water or another liquid, the viscosity of the mixture increases to such a level that it is difficult or impossible to fill an insulin syringe with the mixture. In another embodiment, the viscosity of the heated mixture increases to the level that it may not be deliverable even through needles with the largest diameters commonly used in delivery of insulin.

In one embodiment, the disclosure provides abuse-resistant pharmaceutical formulations comprising an abuse-susceptible active ingredient, at least one polymer, and at least one polysaccharide. In one embodiment, the abuse-susceptible active ingredient is an opioid, preferably an opioid agonist. The polysaccharide can be any known in the art, such as a gum, a cellulose compound, or a combination thereof.

In one embodiment, the disclosure provides orally administrable capsules comprising abuse-resistant pharmaceutical formulations which comprise an opioid, at least one polymer, and at least one polysaccharide. The opioid is preferably an opioid agonist. The polysaccharide can be any known in the art, such as a gum, a cellulose compound, or a combination thereof.

The disclosure provides methods for treating opioid dependence and pain by administering to a human in need thereof a therapeutically effective amount of an abuse-resistant pharmaceutical formulation comprising an opioid, at least one polymer, and at least one polysaccharide. In one embodiment the opioid is preferably an opioid agonist. The polysaccharide can be any known in the art, such as a gum, a cellulose compound, or a combination thereof. The abuse-resistant pharmaceutical formulation can be administered to the human in the form of an orally administrable capsule.

The disclosure provides pharmaceutical formulations that release at least 50 wt % of the abuse-susceptible active ingredient within about 15 minutes via the USP Apparatus III dissolution test; or that release at least 60 wt % of the abuse-susceptible active ingredient within about 15 minutes via the USP Apparatus III dissolution test; or that release at least 70 wt % of the abuse-susceptible active ingredient within about 15 minutes via the USP Apparatus III dissolution test; or that release at least 75 wt % of the abuse-susceptible active ingredient within about 15 minutes via the USP Apparatus III dissolution test; or that release at least 80 wt % of the abuse-susceptible active ingredient within about 15 minutes via the USP Apparatus III dissolution test; or that release at least 85 wt % of the abuse-susceptible active ingredient within about 15 minutes via the USP Apparatus III dissolution test; or that release at least 90 wt % of the abuse-susceptible active ingredient within about 15 minutes via the USP Apparatus III dissolution test; or that release at least 95 wt % of the abuse-susceptible active ingredient within about 15 minutes via the USP Apparatus III dissolution test; or that release at least 98 wt % of the abuse-susceptible active ingredient within about 15 minutes via the USP Apparatus III dissolution test. In embodiments, the pharmaceutical formulation is an orally administrable capsule. In embodiments, the abuse-susceptible active ingredient is an opioid. In embodiments, the abuse-susceptible active ingredient is a buprenorphine compound.

Other features and advantages of the invention will be apparent from the following detailed description and figures, and from the claims.

### DETAILED DESCRIPTION

The abuse-resistant pharmaceutical formulations described herein contain one or more abuse-susceptible active ingredients, one or more polymers, and one or more polysaccharides.

“Active ingredient” or “abuse-susceptible active ingredient” or “abuse-susceptible active” or “active pharmaceutical ingredient” or “active” or “active agent” refers to a pharmaceutical drug that is biologically active, such as opioids, non-opioid analgesics, non-steroidal anti-inflammatory agents, benzodiazepines, barbituates, stimulants, and the like.

In one embodiment, the disclosure provides pharmaceutical formulations comprising (a) an opioid in an amount of about 0.005 wt % to about 25 wt %; (b) at least one polymer in an amount of about 10 wt % to about 90 wt %; and (c) one or more polysaccharides in an amount of about 10 wt % to about 90 wt %. In another embodiment, the pharmaceutical formulation comprises (a) an opioid in an amount of about 0.005 wt % to about 20 wt %; (b) at least one polymer in an amount of about 20 wt % to about 80 wt %; and (c) one or more polysaccharides in an amount of about 10 wt % to about 80 wt %. In another embodiment, the pharmaceutical formulation comprises (a) an opioid in an amount of about 0.005 wt % to about 15 wt %; (b) at least one polymer in an amount of about 30 wt % to about 80 wt %; and (c) one or more polysaccharides in an amount of about 10 wt % to about 70 wt %. In another embodiment, the pharmaceutical formulation comprises (a) an opioid in an amount of about 0.01 wt % to about 5 wt %; (b) at least one polymer in an amount of about 35 wt % to about 75 wt %; and (c) one or more polysaccharides in an amount of about 15 wt % to about 65 wt %. In another embodiment, the pharmaceutical formulation comprises (a) an opioid in an amount of about 0.005 wt % to about 2.0 wt %; (b) at least one polymer in an amount of about 40 wt % to about 70 wt %; and (c) one or more polysaccharides in an amount of about 20 wt % to about 60 wt %. In another embodiment, the pharmaceutical formulation comprises (a) an opioid in an amount of about 0.1 wt % to about 1.2 wt %; (b) at least one polymer in an amount of about 45 wt % to about 65 wt %; and (c) one or more polysaccharides in an amount of about 25 wt % to about 55 wt %. In the pharmaceutical formulations described herein the polysaccharide is a gum, a cellulose compound, or a combination thereof. Each of the terms opioid, polymer, polysaccharide, gum, and cellulose compound are described in more detail herein.

In one embodiment, the disclosure provides pharmaceutical formulations comprising (a) an opioid in an amount of about 0.01 wt % to about 25 wt %; (b) at least one polymer in an amount of about 20 wt % to about 80 wt %; (c) at least one gum in an amount of about 10 wt % to about 60 wt %; and (d) at least one cellulose compound in an amount of about 0.1 wt % to about 50 wt %. In another embodiment, the pharmaceutical formulations comprise (a) an opioid in an amount of about 0.01 wt % to about 5 wt %; (b) at least one polymer in an amount of about 40 wt % to about 75 wt %; (c) at least one gum in an amount of about 20 wt % to about 40 wt %; and (d) at least one cellulose compound in an amount of about 0.1 wt % to about 20 wt %. In another embodiment, the pharmaceutical formulations comprise (a) an opioid in an amount of about 0.05 wt % to about 2.0 wt %; (b) at least one polymer in an amount of about 50 wt % to about 70 wt %; (c) at least one gum in an amount of about 25 wt % to about 35 wt %; and (c) at least one cellulose compound in an amount of about 1 wt % to about 15 wt %. In another embodiment, the pharmaceutical formulations comprise (a) an opioid in an amount of about 0.1 wt % to about 1.2 wt %; (b) at least one polymer in an amount of about 60 wt % to about 65 wt %; (c) at least one gum in an amount of about 28 wt % to about 32 wt %; and (d) at least one cellulose compound in an amount of about 5 wt % to about 10 wt %. Each of the terms opioid, polymer, polysaccharide, gum, and cellulose compound are described in more detail herein.

In one embodiment, the disclosure provides pharmaceutical formulations comprising (a) an opioid in an amount of about 0.01 wt % to about 20 wt %; (b) at least one polymer in an amount of about 10 wt % to about 80 wt %; and (c) at least one gum in an amount of about 10 wt % to about 80 wt %. In another embodiment, the pharmaceutical formulations comprise (a) an opioid in an amount of about 0.01 wt % to about 6 wt %; (b) at least one polymer in an amount of about 35 wt % to about 65 wt %; and (c) at least one gum in an amount of about 40 wt % to about 60 wt %. In another embodiment, the pharmaceutical formulations comprise (a) an opioid in an amount of about 0.05 wt % to about 2.5 wt %; (b) at least one polymer in an amount of about 45 wt % to about 55 wt %; and (c) at least one gum in an amount of about 45 wt % to about 55 wt %. In another embodiment, the pharmaceutical formulations comprise (a) an opioid in an amount of about 0.1 wt % to about 2.0 wt %; (b) at least one polymer in an amount of about 45 wt % to about 55 wt %; and (c) at least one gum in an amount of about 45 wt % to about 55 wt %. In another embodiment, the pharmaceutical formulations comprise (a) an opioid in an amount of about 0.1 wt % to about 1.2 wt %; (b) at least one polymer in an amount of about 48 wt % to about 52 wt %; and (c) at least one gum in an amount of about 48 wt % to about 52 wt %. Each of the terms opioid, polymer, polysaccharide, and gum are described in more detail herein.

The disclosure provides an abuse resistant pharmaceutical formulation comprising (a) about 0.01 wt % to about 10 wt % of a buprenorphine compound; (b) about 10 wt % to about 80 wt % of a poloxamer; and (c) about 25 wt % to about 70 wt % of guar gum. In another embodiment, the disclosure provides a pharmaceutical formulation comprising: (a) about 0.01 wt % to about 5 wt % of a buprenorphine compound; (b) about 35 wt % to about 65 wt % of a poloxamer; and (c) about 40 wt % to about 60 wt % of guar gum. In another embodiment, the disclosure provides a pharmaceutical formulation comprising: (a) about 0.05 wt % to about 2.5 wt % of a buprenorphine compound; (b) about 45 wt % to about 55 wt % of a poloxamer; and (c) about 45 wt % to about 55 wt % of guar gum. In another embodiment, the disclosure provides a pharmaceutical formulation comprising: (a) about 0.1 wt % to about 1.2 wt % of a buprenorphine compound; (b) about 48 wt % to about 52 wt % of a poloxamer; and (c) about 48 wt % to about 52 wt % of guar gum. In these embodiments, the buprenorphine compound is selected from the group consisting of (i) buprenorphine free base; (ii) a pharmaceutically acceptably salt of buprenorphine; (iii) a compound of Formula (I) or a pharmaceutically acceptable salt thereof, (iv) a compound of Formula (II) or a pharmaceutically acceptable salt thereof, (v) a compound of Formula (III) or a pharmaceutically acceptable salt thereof, (vi) a compound of Formula (IV) or a pharmaceutically acceptable salt thereof, or (vii) a compound of Formula (V) or a pharmaceutically acceptable salt thereof. In one embodiment, the buprenorphine compound is buprenorphine hemiadipate. Representative formulations are shown in Example 1.

The disclosure provides an abuse resistant pharmaceutical formulation comprising (a) about 0.01 wt % to about 10 wt % of a buprenorphine compound; (b) about 30 wt % to about 80 wt % of a poloxamer; (c) about 20 wt % to about 50 wt % of guar gum; and (d) about 0.1 wt % to about 20 wt % of a mixture of hydroxypropylmethyl cellulose and carboxymethyl cellulose. In another embodiment, the disclosure provides a pharmaceutical formulation comprising: (a) about 0.01 wt % to about 5 wt % of a buprenorphine compound; (b) about 40 wt % to about 75 wt % of a poloxamer; (c) about 20 wt % to about 40 wt % of guar gum; and (d) about 0.1 wt % to about 20 wt % of a mixture of hydroxypropylmethyl cellulose and carboxymethyl cellulose. In another embodiment, the disclosure provides a pharmaceutical formulation comprising (a) about 0.05 wt % to about 2 wt % of a buprenorphine compound; (b) about 50 wt % to about 70 wt % of a poloxamer; (c) about 25 wt % to about 35 wt % of guar gum; and (d) about 1 wt % to about 15 wt % of a mixture of hydroxypropylmethyl cellulose and carboxymethyl cellulose. In another embodiment, the disclosure provides a pharmaceutical formulation comprising (a) about 0.1 wt % to about 1.2 wt % of a buprenorphine compound; (b) about 60 wt % to about 65 wt % of a poloxamer; (c) about 28 wt % to about 32 wt % of guar gum; and (d) about 5 wt % to about 10 wt % of a mixture of hydroxypropylmethyl cellulose and carboxymethyl cellulose. In these embodiments, the buprenorphine compound is selected from the group consisting of (i) buprenorphine free base; (ii) a pharmaceutically acceptably salt of buprenorphine; (iii) a compound of Formula (I) or a pharmaceutically acceptable salt thereof, (iv) a compound of Formula (II) or a pharmaceutically acceptable salt thereof, (v) a compound of Formula (III) or a pharmaceutically acceptable salt thereof, (vi) a compound of Formula (IV) or a pharmaceutically acceptable salt thereof, or (vii) a compound of Formula (V) or a pharmaceutically acceptable salt thereof. In one embodiment, the buprenorphine compound is buprenorphine hemiadipate. Representative formulations are shown in Example 2.

In one embodiment, the disclosure provides capsules that release at least 50 wt % of the opioid within 30 minutes via the USP Apparatus III dissolution test. In one embodiment, the disclosure provides capsules that release at least 75 wt % of the opioid within 30 minutes via the USP Apparatus III dissolution test. In one embodiment, the disclosure provides capsules that release at least 80 wt % of the opioid within 30 minutes via the USP Apparatus III dissolution test. In one embodiment, the disclosure provides capsules that release at least 85 wt % of the opioid within 30 minutes via the USP Apparatus III dissolution test. In one embodiment, the disclosure provides capsules that release at least 90 wt % of the opioid within 30 minutes via the USP Apparatus III dissolution test. In one embodiment, the disclosure provides capsules that release at least 95 wt % of the opioid within 30 minutes via the USP Apparatus III dissolution test. In one embodiment, the disclosure provides capsules that release at least 98 wt % of the opioid within 30 minutes via the USP Apparatus III dissolution test. In embodiments, the capsules contain an abuse-susceptible active agent instead of an opioid.

In one embodiment, the disclosure provides capsules that release at least 50 wt % of the opioid within 15 minutes via the USP Apparatus III dissolution test. In one embodiment, the disclosure provides capsules that release at least 55 wt % of the opioid within 15 minutes via the USP Apparatus III dissolution test. In one embodiment, the disclosure provides capsules that release at least 60 wt % of the opioid within 15 minutes via the USP Apparatus III dissolution test. In one embodiment, the disclosure provides capsules that release at least 65 wt % of the opioid within 15 minutes via the USP Apparatus III dissolution test. In one embodiment, the disclosure provides capsules that release at least 70 wt % of the opioid within 15 minutes via the USP Apparatus III dissolution test. In one embodiment, the disclosure provides capsules that release at least 75 wt % of the opioid within 15 minutes via the USP Apparatus III dissolution test. In one embodiment, the disclosure provides capsules that release at least 80 wt % of the opioid within 15 minutes via the USP Apparatus III dissolution test. In one embodiment, the disclosure provides capsules that release at least 85 wt % of the opioid within 15 minutes via the USP Apparatus III dissolution test. In one embodiment, the disclosure provides capsules that release at least 90 wt % of the opioid within 15 minutes via the USP Apparatus III dissolution test. In one embodiment, the disclosure provides capsules that release at least 95 wt % of the opioid within 15 minutes via the USP Apparatus III dissolution test. In one embodiment, the disclosure provides capsules that release at least 98 wt % of the opioid within 15 minutes via the USP Apparatus III dissolution test. In embodiments, the capsules contain an abuse-susceptible active agent instead of an opioid.

In one embodiment, the disclosure provides capsules that release about 50 wt % to 100 wt % of the opioid within 15 minutes via the USP Apparatus III dissolution test. In one embodiment, the disclosure provides capsules that release about 75 wt % to 100 wt % of the opioid within 15 minutes via the USP Apparatus III dissolution test. In one embodiment, the disclosure provides capsules that release about 80 wt % to 100 wt % of the opioid within 15 minutes via the USP Apparatus III dissolution test. In one embodiment, the disclosure provides capsules that release about 85 wt % to 100 wt % of the opioid within 15 minutes via the USP Apparatus III dissolution test. In one embodiment, the disclosure provides capsules that release about 90 wt % to 100 wt % of the opioid within 15 minutes via the USP Apparatus III dissolution test. In one embodiment, the disclosure provides capsules that release about 95 wt % to 100 wt % of the opioid within 15 minutes via the USP Apparatus III dissolution test. In one embodiment, the disclosure provides capsules that release about 98 wt % to 100 wt % of the opioid within 15 minutes via the USP Apparatus III dissolution test. In embodiments, the capsules contain an abuse-susceptible active agent instead of an opioid.

The relatively high viscosity pharmaceutical formulations described here make it difficult to extract the opioid through the use of needles. In one embodiment, the opioid is an opioid agonist. The term opioid, which is synonymous with “opiates” for purposes of this disclosure, includes opioid agonists and opioid antagonists. The opioid agonist can be a full opioid agonist or a partial opioid agonist. The abuse-resistant pharmaceutical formulations described herein can contain one or more opioids. In one embodiment, the pharmaceutical formulation contains an opioid agonist. In one embodiment, the pharmaceutical formulation contains an opioid agonist and an opioid antagonist. It has been discovered that the relatively high viscosity of the pharmaceutical formulations make it difficult to withdraw and inject the opioid contained therein. Because the excipients used in the pharmaceutical formulation prevent abuse, it is generally unnecessary to include an opioid antagonist in the formulation.

The opioid used in the pharmaceutical formulations and methods described herein can be any opioid known in the art, including natural opiates, synthetic opioids, and semi-synthetic opioids. Exemplary opioids include buprenorphine, adulmine, alfentanil, allocryptopine, allylprodine, alphaprodine, anileridine, aporphine, benzylmorphine, berberine, bicuculine, bicucine, bezitramide, bulbocaprine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tapentadol, tilidine, tramadol, or a pharmaceutically acceptable salt of any of the foregoing, or a pharmaceutically acceptable prodrug of any of the foregoing.

In one embodiment, the opioid is buprenorphine, morphine, hydrocodone, oxycodone, methadone, meperidine, hydromorphone, fentanyl, codeine, or a pharmaceutically acceptable salt of any of the foregoing, or a pharmaceutically acceptable prodrug of any of the foregoing. In one embodiment, the opioid is morphine or a pharmaceutically acceptable salt thereof. In one embodiment, the opioid is hydrocodone or a pharmaceutically acceptable salt thereof. In one embodiment, the opioid is oxycodone or a pharmaceutically acceptable salt thereof. In one embodiment, the opioid is methadone or a pharmaceutically acceptable salt thereof. In one embodiment, the opioid is meperidone or a pharmaceutically acceptable salt thereof. In one embodiment, the opioid is hydromorphone or a pharmaceutically acceptable salt thereof. In one embodiment, the opioid is fentanyl or a pharmaceutically acceptable salt thereof. In one embodiment, the opioid is codeine or a pharmaceutically acceptable salt thereof.

In one embodiment, the opioid is a buprenorphine compound. The term “buprenorphine compound” includes buprenorphine free base; pharmaceutically acceptable salts (e.g., HCl) of buprenorphine; and buprenorphine prodrugs (e.g., in the form of the free base or pharmaceutically acceptable salt). In one embodiment, the buprenorphine compound is buprenorphine free base. In one embodiment, the buprenorphine compound is a pharmaceutically acceptable salt (e.g., HCl) of buprenorphine. In one embodiment, the buprenorphine prodrug is a compound of Formula (I), Formula (II), Formula (III), Formula (IV), or Formula (V), each of which are described herein and in U.S. Pat. No. 7,964,610, the disclosure of which is incorporated by reference herein in its entirety.

In one embodiment, the buprenorphine compound is a compound of Formula (I) or pharmaceutically acceptable salt thereof:

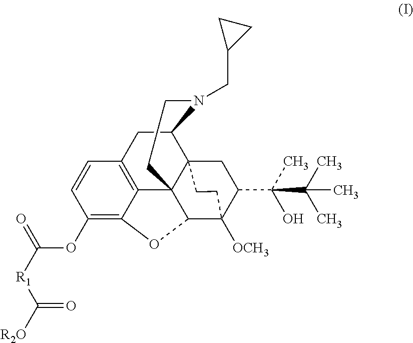

wherein R₁ is (1) a C₁-C₁₀ straight-chain or branched alkylene or alkyl moiety, optionally substituted with a aromatic ring, e.g., a carbocyclic or heterocyclic aromatic ring; (2) a —(CH₂)ₚCH═CH(CH₂)ₚ— moiety in which each p is independently an integer from 0 to 4; or (3) a —(CH₂)ₙX(CH₂)ₙ— moiety in which each n is an integer from 0 to 2, X is O, S, NH, a 5-membered ring represented by Structure 2 (below) having 1,2-(structure 2A below), 1,3-(2B), or 1,4-substitution (2C) in which Y is O, S or NH, a benzene ring represented by Structure 3 (below) having 1,2-(3A), 1,3-(3B), or 1,4-substitution (3C) or a 5-, 6-, 7- or 8-membered alkyl ring, as represented by Structure 4 (below). In instances in which X is a 5-, 6-, 7- or 8-membered alkyl ring, all positional isomers of each respective ring systems can be utilized, e.g., 1,2- and 1,3-substitiuton for the 5-membered ring. In Formula (I), R₂ is H or a C₁-C₆ straight-chain or branched alkyl.

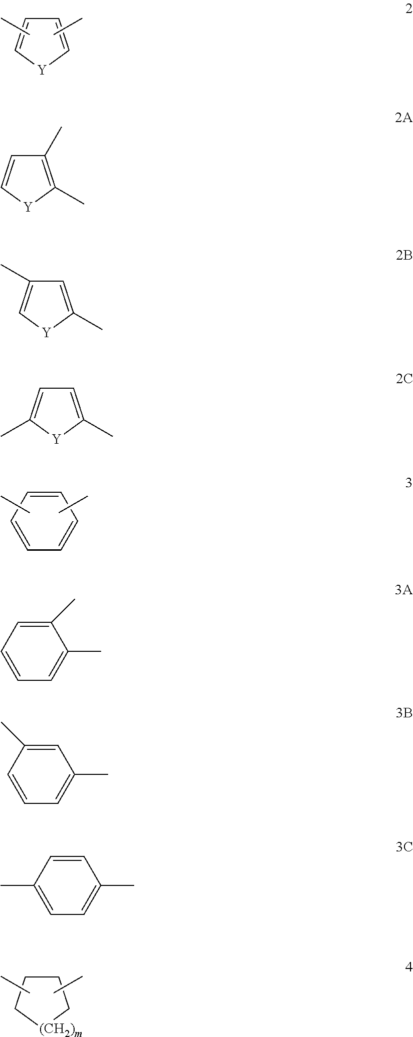

Some examples of C₁-C₁₀ straight-chain or branched alkyl moieties include, e.g., —CH₂—, —CH₂CH₂—, —CH₂CH₂CH₂—, —CH₂CH₂CH₂CH₂CH₂- and Structure 5, 6, 7, and 8 below.

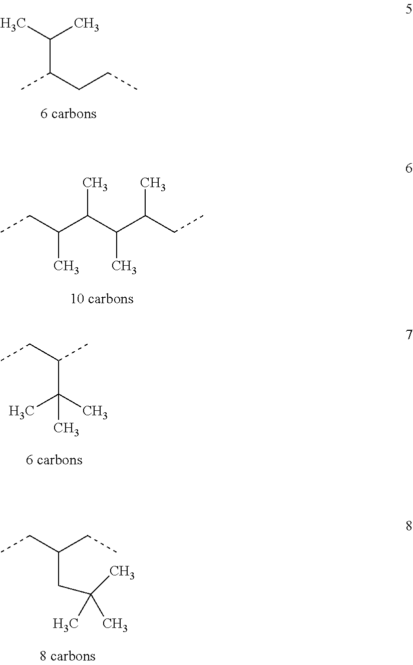

Some examples of C₁-C₁₀ straight-chain or branched alkyl moieties substituted with an aromatic ring include Structure 9, 10, 11, and 12 below.

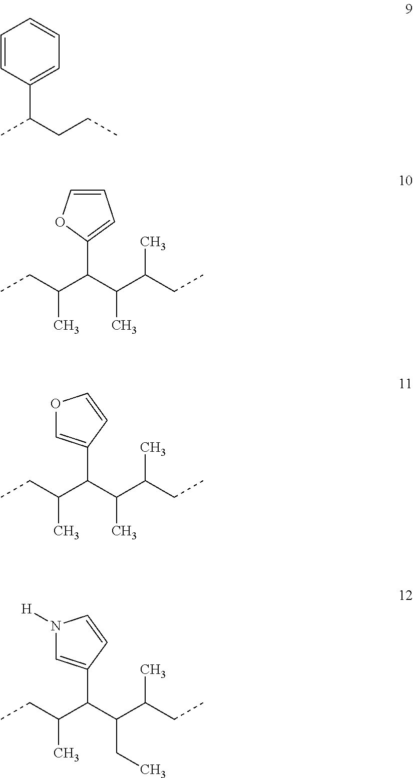

The aromatic ring can be, e.g., a single ring or a fused ring. The aromatic ring can be carbocyclic ring (e.g., a benzene ring or a naphthalene ring system), a heterocyclic ring (e.g., a thiophene derivative, a furan derivative, or a pyrrole derivative) or a fused carbocyclic and hetercyclic ring.

In specific embodiments, R₁ is —CH₂CH₂—, —CH₂CH₂CH₂—, —CH₂CH₂CH₂CH₂—, —CH₂CH(CH₃)CH₂—, —CH₂C(CH₃)₂CH₂—, —CH₂OCH₂—, —CH₂SCH₂—, —CH₂NHCH₂—, or —CH₂N(COOCH₂Ph)CH₂—.

In instances in which R₂ is a C₁-C₆ straight-chain or branched alkyl moiety, R₂ can be, e.g., methyl, ethyl, propyl, isopropyl, butyl, isobutyl, sec-butyl, t-butyl, amyl, isoamyl, 1,2-dimethylpropyl, 1,1-dimethylpropyl, pentyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2,2-trimethylpropyl, and 1,1,2-trimethylpropyl.

When R₂ is H, the buprenorphine compound is a compound of Formula (II) or pharmaceutically acceptable salt thereof.

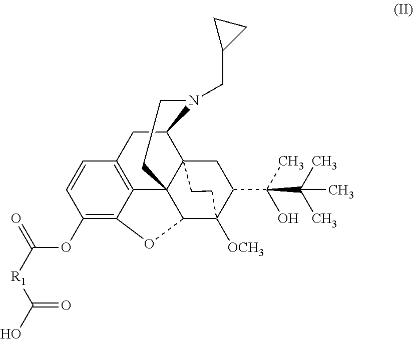

In such instances, in Formula (II), R₁ is (1) a C₁-C₁₀ straight-chain alkylene or alkyl moiety; (2) a C₁-C₈ straight-chain alkylene moiety substituted with from 1 to 4 methyl groups or a carbocyclic aromatic ring, e.g., a phenyl group; or (3) a —(CH₂)ₚCH═CH(CH₂)ₚ— moiety in which each p is independently an integer from 0 to 3.

In some embodiments, compounds or salts of Formula (II) are those for which R₁ is C₂-C₅ straight-chain alkyl, e.g., —CH₂CH₂CH₂—, —CH₂CH₂CH₂CH₂—, —CH₂CH₂CH₂CH₂CH₂—, —CH₂CH(CH₃)CH₂— or —CH₂C(CH₃)₂CH₂—.

In other embodiments, the buprenorphine compound is a compound of Formula (III) or a pharmaceutically acceptable salt thereof:

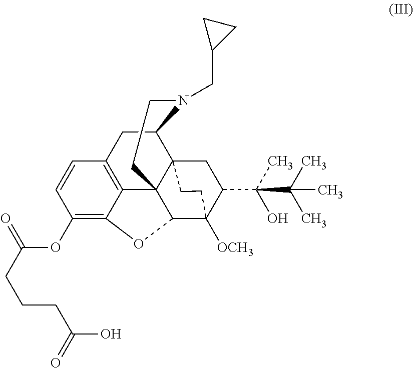 [SMILES: CO[C@]12CC[C@@]3(C[C@@H]1[C@](C)(O)C(C)(C)C)[C@H]1Cc4ccc(OC(=O)CCCC(=O)O)c5c4[C@@]3(CCN1CC1CC1)[C@H]2O5]

In other embodiments, the buprenorphine compound is a compound of Formula (N) or a pharmaceutically acceptable salt thereof:

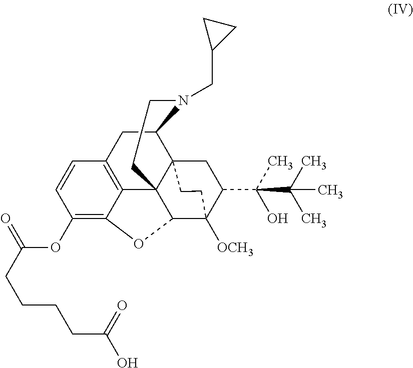 [SMILES: CO[C@]12CC[C@@]3(C[C@@H]1[C@](C)(O)C(C)(C)C)[C@H]1Cc4ccc(OC(=O)CCCCC(=O)O)c5c4[C@@]3(CCN1CC1CC1)[C@H]2O5]

When R₂ is H, and R₁ is —(CH₂)ₙX(CH₂)ₙ—, the buprenorphine prodrug is a compound of Formula (V) or a pharmaceutically acceptable salt thereof:

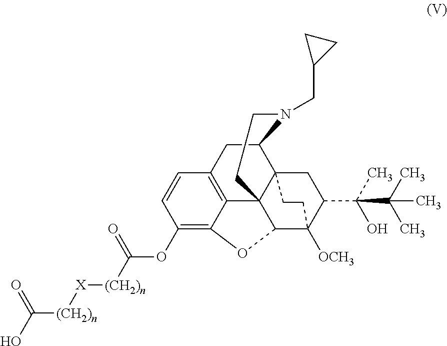

In such instances, —(CH₂)ₙX(CH₂)ₙ— can be any of the moieties described above. In specific embodiments, n is 1 in each occurrence and X is S, NH, N(COOCH₂Ph) or O.

The buprenorphine compound described herein can be made by any method known in the art, including the methods described in U.S. Pat. No. 7,964,610, the disclosure of which is incorporated herein by reference in its entirety.

In other embodiments, the pharmaceutical formulation may comprise an opioid agonist and an opioid antagonist. The opioid agonist can be any opioid agonist described herein. The opioid antagonist can be naloxone, naltrexone, nalmefene, nalorphine, nalbuphine, naloxoneazinen, methylnaltrexone, ketylcyclazocine, norbinaltorphimine, naltrindol, 6-beta-naloxol, 6-b-naltrexol, or a pharmaceutically acceptable salt of any one of the foregoing. In one embodiment, the pharmaceutical formulation may contain buprenorphine and naloxone; or tilidine and naloxone; or hydrocodone and naltrexone; or oxycodone and naltrexone; or other therapeutically effective combinations of opioid agonists and opioid antagonists known in the art. Because the pharmaceutical formulations described herein are abuse resistant, it is generally not necessary to include an opioid antagonist in the formulations.

The opioid is present in the pharmaceutical formulations described herein in a therapeutically effective amount, an amount which may be dependent on the use of the pharmaceutical formulation, e.g., for treating opioid dependence or pain. In one embodiment, the opioid is present in the pharmaceutical formulation in an amount of about 0.005 to about 25 wt %. In another embodiment, the opioid is present in the pharmaceutical formulation in an amount of about 0.005 to about 20 wt %. In another embodiment, the opioid is present in the pharmaceutical formulation in an amount of about 0.05 to about 15 wt %. In another embodiment, the opioid is present in the pharmaceutical formulation in an amount of about 0.05 to about 10 wt %. In another embodiment, the opioid is present in the pharmaceutical formulation in an amount of about 0.05 to about 7 wt %. In another embodiment, the opioid is present in the pharmaceutical formulation in an amount of about 0.05 to about 5 wt %. In another embodiment, the opioid is present in the pharmaceutical formulation in an amount of about 0.05 to about 2 wt %. In another embodiment, the opioid is present in the pharmaceutical formulation in an amount of about 0.05 to about 1.5 wt %. In another embodiment, the opioid is present in the pharmaceutical formulation in an amount of about 0.1 to about 1.2 wt %.

In certain embodiments, the abuse-susceptible active agent is selected from the group consisting of a non-opioid analgesic, non-steroidal anti-inflammatory agents, benzodiazepinres, barbituates, stimulants, and mixtures thereof.

In one embodiment, the abuse-susceptible active agent a selected from a non-opioid analgesic.

In another embodiment, the abuse-susceptible active agent is selected from a non-steroidal anti-inflammatory agent consisting of aspirin, celecoxib, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam, pharmaceutically acceptable salts and mixtures thereof.

In another embodiment, the abuse-susceptible active agent is benzodiazepines, selected from the group consisting of alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, pharmaceutically acceptable salts and mixtures thereof.

In another embodiment, the abuse-susceptible active agent a barbiturate, selected from the group consisting of amobarbital, aprobarbotal, butabarbital, butalbital, methohexital, mephobarbital, metharbital, pentobarbital, phenobarbital, secobarbital and pharmaceutically acceptable salts thereof and mixtures thereof.

In another embodiment, the abuse-susceptible active agent a stimulant, selected from the group consisting of amphetamines, dextroamphetamine resin complex, dextroamphetamine, methamphetamine, methylphenidate, pharmaceutically acceptable salts and mixtures thereof.

The abuse-suscetpible active ingredients are formulated as an abuse-resistant, relatively high viscosity, pharmaceutical formulation using at least one polymer in the amounts described above. Polymers useful in the pharmaceutical formulations described herein include a polyoxypropylene, a polyoxyethylene, a polyglycolic acid, a polylactic acid, a polydioxane, a polyoxalate, a poly(alpha-ester), a polyanhydride, a polyacetate, a polycaprolactone, a poly(orthoester), a polyamino acid, a polyaminocarbonate, a polyurethane, a polycarbonate, a polyamide, a poly(alkyl cyanoacrylates), or a mixture of two or more of the foregoing, or a copolymer of any of the foregoing, or a block copolymer of two or more of the foregoing. The polymers are preferably in the form of a block copolymer or a triblock copolymer.

In one embodiment, the polymer is a copolymer of polyoxypropylene and polyoxyethylene or a block copolymer of polyoxypropylene and polyoxyethylene. In one embodiment, the polymer is a triblock copolymer of polyoxypropylene and polyoxyethylene. In one embodiment the polymer is a poloxamer. Poloxamers are nonionic triblock copolymers in which a central hydrophobic polyoxypropylene polymer is flanked on both sides by a polyoxyethylene polymer. Poloxamers have the following general formula and structure:

HO—[—CH₂—CH₂—O—]ₐ—[—CH(CH₃)—CH₂—O—]_{b}—[—CH₂—CH₂—O—]ₐ—H

wherein a=2-130, and b=15-67. Exemplary poloxamers include poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, or a combination of two or more thereof. In one embodiment, the poloxamer is poloxamer 122, poloxamer 123, poloxamer 124, poloxamer 181, poloxamer 182, poloxamer 183, poloxamer 184, poloxamer 185, poloxamer 188, or a combination of two or more thereof. In one embodiment, the poloxamer is poloxamer 122, poloxamer 123, poloxamer 124, or a combination of two or more thereof. In one embodiment, the poloxamer is poloxamer 124. For the numbers following the term poloxamer, the first two digits×100 give the molecular mass of the central polyoxypropylene polymer, and the last digit×10 gives the percentage of the polyoxyethylene polymer content. For example, poloxamer 407 is a poloxamer with a polyoxypropylene molecular mass of 4,000 g/mol and a 70% polyoxyethylene content. Poloxamers are available by the tradenames PLURONICS® (BASF Corporation), SYNPERONICS® (Croda International), and KOLLIPHOR® (BASF SE).

In one embodiment, the only polymer contained in the pharmaceutical formulations described herein is a poloxamer or a mixture of poloxamers, and the pharmaceutical formulations do not contain any other polymers. In one embodiment, this may be described as a pharmaceutical formulation comprising a polymer consisting of at least one poloxamer.

The abuse-susceptible active ingredients are formulated as an abuse-resistant, relatively high viscosity, pharmaceutical formulation using at least one gum in the amounts described herein. Gums that are useful in the pharmaceutical formulations herein include guar gum, locust bean gum, tara gum, dextran, carrageenan, xanthan gum, gellan gum, chitan, chitosan, acacia gum, gum arabic, tragacanth gum, karaya gum, mesquite gum, pectin, gum levan, xylan, pullulan, mannan, mannoglucan, carob bean gum, or a combination of two or more thereof. In one embodiment the gum is carrageenan, dextran, gum arabic, tragacanth gum, pectin, karaya gum, xanthan gum, guar gum, or a combination of two or more thereof. In one embodiment, the gum is xanthan gum, guar gum, or a combination thereof. In one embodiment, the gum is guar gum.

The abuse-susceptible active ingredients are formulated as an abuse-resistant, relatively high viscosity, pharmaceutical formulation optionally using at least one cellulose compound in the amounts described herein. Cellulose compounds that are useful in the pharmaceutical formulations described herein include alkyl cellulose, hydroxyalkyl cellulose, carboxyalkyl cellulose, or combinations of two or more thereof. In one embodiment, the cellulose compound is methyl cellulose, ethyl cellulose, hydroxypropyl cellulose, hydroxypropylmethyl cellulose (HPMC), ethylmethyl cellulose, carboxymethyl cellulose (CMC), ethylhydroxyethyl cellulose, or a combination of two or more thereof. In one embodiment, the cellulose compound is a mixture of hydroxypropylmethyl cellulose and carboxymethyl cellulose. In one embodiment, the cellulose compound is hydroxypropylmethyl cellulose. In one embodiment, the cellulose compound is carboxymethyl cellulose. When two cellulose compounds are used in the formulations described herein, they can be present in a ratio of 1:10 to 10:1, or a ratio of 1:5 to 5:1, or a ratio of 2.5:1 to 1:2.5, or a ratio of 1:1.

The abuse-resistant pharmaceutical formulations described herein may optionally contain other excipients that enhance the clinical or chemical properties of the formulation. For example, the formulation may further comprise antioxidants, surfactants, buffers, preservatives, or other fillers. In one embodiment, the abuse-resistant formulations do not contain any additional excipients.

The abuse-resistant pharmaceutical formulations described herein may be administered by any method known in the art. In one embodiment, the pharmaceutical formulations are administered orally. The terms “oral,” “orally,” “oral administration,” and “orally administered” refer to administration or delivery of the pharmaceutical formulation via the gastro-intestinal tract, i.e., where the human swallows the pharmaceutical formulation, or the capsule containing the pharmaceutical formulation, such that the abuse-susceptible active ingredient is released into the gastrointestinal tract.

In one embodiment, the abuse-susceptible active ingredient is released rapidly into the use environment. By “released rapidly” is meant within less than 2 hours after administration, or even less than 1 hour after administration, less than 30 minutes after administration, or even less than 15 minutes after administration. In one embodiment, at least 80 wt % of the abuse-susceptible active ingredient is released within 30 minutes following administration to an in vitro dissolution test using a Dissolution Apparatus 3, equipped with reciprocating cylinders operating a stroke rate of 30 min⁻¹ in pH 2 phosphate buffer solution. In still another embodiment, 80 wt % of the abuse-susceptible active ingredient is released within 15 minutes using the same in vitro test protocol.

When the pharmaceutical formulations are orally administered, they may be placed inside a capsule. Capsule technology is well known in the art. The capsule can be a hard capsule or a soft capsule. The capsule may be made, for example, from gelatin, pullulan, hypromellose, or a combination of two or more thereof. In one embodiment, capsules are filled with the relatively viscous pharmaceutical formulations described herein. In one embodiment, the pharmaceutical formulation has a viscosity greater than 2,000 cp, or greater than 5,000 cp, or greater than 10,000 cp, or greater than 15,000 cp, or greater than 20,000 cp. In embodiments, the pharmaceutical formulation has a viscosity from about 2,000 cp to about 75,000 cp; or from about 5,000 cp to about 70,000 cp; or from about 10,000 to about 60,000 cp; or from about 10,000 cp to about 50,000 cp; or from about 5,000 cp to about 25,000 cp; or about 5,000 cp to about 10,000 cp.

The abuse-resistant pharmaceutical formulations described herein can be used for treating opioid dependence and treating pain.

In one embodiment, the methods are for treating opioid dependence. For purposes of this disclosure, the term “opioid dependence” encompasses each of the terms opioid dependence, opioid use disorder, opioid abuse, opioid addiction, and opioid withdrawal symptoms. The term “opioid withdrawal symptoms” refer to the symptoms that occur when a human stops using opioids or reduces their opioid intake, and may include one or more of the following symptoms: agitation, anxiety, irritability, fatigue, muscle aches, increased tearing, insomnia, runny nose, sweating, yawning, abdominal cramping, diarrhea, dilated pupils, goose bumps, nausea, and vomiting. In some embodiments, the opioid being abused is heroin or a prescription pain medication (e.g., oxycodone, hydrocodone, morphine, fentanyl, codeine). The treatment of opioid dependence may be a short-term treatment of opioid dependence or maintenance treatment of opioid dependence. For the treatment of opioid dependence, the opioid used in the pharmaceutical formulations described herein may preferably be a buprenorphine compound, such as those described herein.

The methods described herein include methods for treating pain. The pain can be acute pain or chronic pain, and the treatment can be short-term treatment or chronic treatment. For the treatment of pain, the opioid used in the pharmaceutical formulations described herein may preferably be buprenorphine, oxycodone, hydrocodone, morphine, fentanyl, or codeine.

For the treatment of opioid dependence and pain, the dosage, toxicity and therapeutic efficacy of the pharmaceutical formulations described herein can be determined by reference to standard pharmaceutical procedures in cell cultures and/or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. The data obtained from animal studies can be used in formulating a range of dosage for use in humans. The dosage of each pharmaceutical formulation lies preferably within a range of circulating concentrations that include the ED50 with little or no toxicity. The dosage may vary within this range depending upon the dosage form employed and the route of administration utilized.

A “therapeutically effective amount” is an amount sufficient to effect beneficial or desired results for treating the disease, such as opioid dependence or pain. In one embodiment, the therapeutically effective amount of opioid, such as an opioid, may be from about 0.01 mg to about 1,000 mg per day; or from about 0.1 mg to about 500 mg per day; or from about 0.1 mg to about 100 mg per day; or from about 1 mg to about 55 mg per day; or from about 1 mg to about 50 mg; or from about 2 mg to about 45 mg; or from about 2 mg to about 35 mg per day; or from about 3 mg to about 30 mg; or from about 5 mg to about 25 mg; or from about 10 mg to about 30 mg per day. A therapeutic amount is one that achieves the desired therapeutic effect and may be determined or estimated by referencing the dosages used in commercially available products. An effective amount can be administered in one or more administrations, applications or dosages. For example, the compositions can be administered once or twice per day. The skilled artisan will appreciate that certain factors may influence the dosage and timing required to effectively treat a human, including but not limited to the severity of the opioid dependence or pain, previous treatments, the general health and/or age of the human, and other diseases present. Moreover, treatment of a human with a therapeutically effective amount of the compositions described herein can include a single treatment or a series of treatments.

### EXAMPLES

The following examples are for purposes of illustration only, and are not intended to limit the scope of the disclosure or claims.

### Example 1

In preparing Example 1D, a mixture containing 3.2 wt % buprenorphine hemiadipate HCl, 48.4 wt % Poloxamer 124, and 48.4 wt % guar gum was added to a container, and heated in an oven until 55±5° C. The mixture was then blended on a Silverson Laboratory High Shear Mixer for less than 10 minutes until the mixture was deemed homogenized by visual assessment. The resulting material was degassed under vacuum then placed into gelatin capsules using the semi-automated HiBar Capsule Filling Machine, which were then banded with a clear gelatin band and allowed to dry for 16 hours until further processing. With reference to the preparation for Example 1D described above and in reference to Table 1 below, Examples 1A-F were prepared to produce capsules containing 2.5 mg, 5 mg, 10 mg, 20 mg, 30 mg, and 50 mg of buprenorphine hemiadipate HCl.

TABLE 1

| Component | A | B | C | D | E | F |
|---|---|---|---|---|---|---|
| buprenorphine hemiadipate | 2.5 | 5 | 10 | 20 | 30 | 50 |
| HCl (dosage in mg) | | | | | | |
| buprenorphine hemiadipate | 0.4 | 0.8 | 1.6 | 3.2 | 4.8 | 8.0 |
| HCl (wt %) | | | | | | |
| poloxamer 124 (wt %) | 49.8 | 49.6 | 49.2 | 48.4 | 47.6 | 46.0 |
| guar gum (wt %) | 49.8 | 49.6 | 49.2 | 48.4 | 47.6 | 46.0 |
| Total (wt %) | 100.0 | 100.0 | 100.0 | 100.0 | 100.0 | 100.0 |
| formulation weight in mg | 625 | 625 | 625 | 625 | 625 | 625 |

### Example 2

Examples 2G-L were prepared to produce capsules containing 2.5 mg, 5 mg, 10 mg, 20 mg, 30 mg and 50 mg of buprenorphine hemiadipate HCl, as shown in Table 2.

TABLE 2

| Component | G | H | I | J | K | L |
|---|---|---|---|---|---|---|
| buprenorphine hemiadipate | 2.5 | 5 | 10 | 20 | 30 | 50 |
| HCl (dosage in mg) | | | | | | |
| buprenorphine hemiadipate | 0.4 | 0.8 | 1.6 | 3.2 | 4.8 | 8.0 |
| HCl (wt %) | | | | | | |
| poloxamer 124 (wt %) | 61.9 | 61.6 | 61.1 | 60.0 | 58.9 | 56.8 |
| guar gum (wt %) | 30.2 | 30.1 | 29.8 | 29.3 | 28.8 | 27.7 |
| hydroxypropylmethyl | 3.5 | 3.5 | 3.5 | 3.5 | 3.5 | 3.5 |
| cellulose (wt %) | | | | | | |
| carboxymethyl cellulose | 4.0 | 4.0 | 4.0 | 4.0 | 4.0 | 4.0 |
| (wt %) | | | | | | |
| Total (wt %) | 100.0 | 100.0 | 100.0 | 100.0 | 100.0 | 100.0 |
| Formulation weight in mg | 625 | 625 | 625 | 625 | 625 | 625 |

### Example 3

Abuse resistance testing was performed using the capsules of Examples 1 and 2 in the following procedure using the following solvents: (1) ambient water, (2) hot water and (3) simulated liquor, i.e. 60:40 water:ethanol. A capsule was placed into a mortar and up to 6 mL of the solvent was added. The capsule was then ground with a pestle for 5 minutes. This resulted in a light brown viscous liquid that could not be drawn into a syringe. In instances where residual liquid was present it was drawn into a syringe and a needle was attached. The liquid did not flow freely through the needle, and the needle was forced off the syringe, without delivery of the liquid. This demonstrates the abuse resistance of the capsules.

### Example 4

Abuse resistant formulations are specifically designed to deter or hinder the unlawful extraction and misuse of controlled drugs, like opioids, from the drug product (formulation). The difficulty in extraction can result in challenges during dissolution analysis. As a result, issues can occur when determining the release profile of an opioid from an abuse-resistant formulation. For immediate release formulations, the generation of a release profile can be particularly challenging using standard dissolution testing. The standard approach for tablet and capsule analysis uses Apparatus I/II (basket/paddle) detailed in the Pharmacopeia. These types of dissolution equipment are more suited to standard instant and sustained release dosage forms. Apparatus III uses a reciprocating cylinder that sits within a larger vessel. The agitation is similar to that of disintegration equipment; however, the design allows for repeatable sampling to be performed for single dose units.

The dissolution testing was performed to evaluate the percentage release of buprenorphine hemiadipate under acidic gastric conditions. This was in keeping with the immediate release required for the formulation. A dog pK study indicated that in-vivo buprenorphine hemiadipate released immediately, indicating that dissolution testing using USP Apparatus I/II was not representative of the formulation performance when ingested.

Abuse resistance testing was performed using the capsules of Examples 1 and 2. The materials and methods used for dissolution testing of the abuse-resistant formulations are shown in Table 3. For sample analysis, an Agilent HPLC quantitation method used, equipped with Phenomenex Kinetex C18, 2.6 μm, 50 mm×4.6 mm Column, maintained at 40° C., with a flow rate of 1.0 mL/min, an injection volume of 50 μL. The disintegration equipment was from Copley (baskets suitable for larger capsule size) with no discs; however, sinkers were used.

TABLE 3

| Agilent Dissolution | Apparatus and speeds |
|---|---|
| Basket (USP Apparatus I) | 75 rpm |
| Paddles (USP Apparatus II) | 75 rpm |
| Reciprocal (USP Apparatus III) | 30 dpm |
| Copley Disintegration Bath dip rate | 30 dpm |
| Bath temperature | 37° C. ± 0.5° C. |
| Sample volume removed | 5 mL |
| Volume of medium Apparatus I and | 500 mL and 900 mL |
| Apparatus II | |
| Volume of medium for Disintegration | 600 mL |
| Volume of medium Apparatus III | 250 mL |
| Dissolution Medium | 50 mM Phosphate |
| | Buffer pH 2.0 |

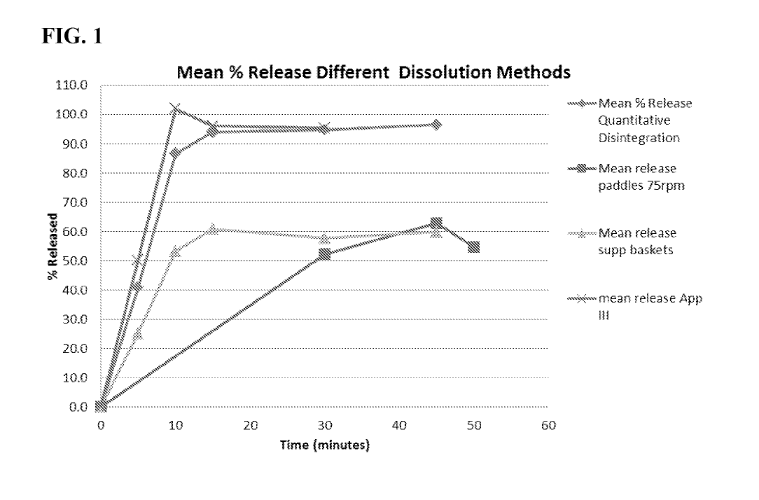

Disintegration analysis was performed using the USP Disintegration Test, Chapter 701 (Aug. 1, 2008) and, as shown in FIG. 1, indicated that the capsule would disperse fully in approximately 15 minutes. Quantitative analysis by sampling at time points during the disintegration test was performed to ascertain the buprenorphine hemiadipate release using this form of agitation. The data obtained were consistent with the results from the dog pK study. This suggested that the hydrodynamics used in the disintegration test was better suited to the physical dispersion of the formulation. Both the USP Apparatus I and II utilize rotational agitation where disintegration uses a reciprocating (dipping) action. The USP Apparatus III dissolution system also uses a vertical reciprocating movement. This design of dissolution was further evaluated for its suitability.

The reciprocating action of the USP Apparatus III dissolution was found to be the significant factor in the release of buprenorphine hemiadipate from the abuse deterrent formulation. The more aggressive dipping action was essential to the dispersion of the drug product and therefore the solubility of buprenorphine hemiadipate. The resultant in-vitro dissolution profile was consistent with the data obtained in the dog pK study indicating that the required immediate release profile was obtained and a corresponding in-vivo/in-vitro correlation implied.

Buprenorphine hemiadipate was known to be highly soluble in aqueous solutions and therefore should readily dissolve in the proposed dissolution medium (pH 2, 50 mM Phosphate Buffer in water). Additional development of the dissolution method conditions did not improve the release of buprenorphine hemiadipate. This included the addition of enzyme to the medium to aid in the disintegration of the formulation to allow for the release of buprenorphine hemiadipate into the solution. The physical dispersion of the formulation into the dissolution medium was examined by increasing the paddle speed and altering the sinker and basket designs. The sinkers used initially were of USP design (5 Spirals). It was thought that the helix may be too tight and due to the gum generated, the formulation would not disperse. A simple stainless steel coil was fashioned to weight the capsule down in the medium. Similarly a more open basket design was used during the dissolution using USP Apparatus I. Neither the chemical nor physical alterations improved the in-vitro release characteristics.

This demonstrated the suitability of USP Apparatus III as an in-vitro technique for predicting in-vivo performance of the formulations described herein. Additional considerations for dissolution development using Apparatus III are the sieve size for the top and bottom of the inner vessel. This can have an impact on the flow of the medium over the formulation and therefore the release of the drug substance.

### Example 5

In vitro release testing was performed with the capsules of Examples 1 and 2 using the following protocol. Six capsules were tested via Dissolution Apparatus 3, equipped with reciprocating cylinders operating at a stroke rate of 30 min⁻¹ in pH 2 phosphate buffer. Buprenorphine hemiadipate HCl was released (˜ 80%) within 15 minutes.

### Embodiments

The following embodiments are for purposes of illustration and exemplification, and are not intended to limit the scope of the claims or the disclosure.

Embodiment 1. A pharmaceutical formulation comprising: (a) about 0.01 wt % to about 10 wt % of an opioid; (b) about 10 wt % to about 80 wt % of a polymer selected from the group consisting of a copolymer of polyoxypropylene and polyoxyethylene, and a block copolymer of polyoxypropylene and polyoxyethylene; and (c) about 25 wt % to about 70 wt % of a gum.

Embodiment 2. A pharmaceutical formulation comprising: (a) about 0.01 wt % to about 5 wt % of an opioid; (b) about 35 wt % to about 65 wt % of a polymer selected from the group consisting of a copolymer of polyoxypropylene and polyoxyethylene, and a block copolymer of polyoxypropylene and polyoxyethylene; and (c) about 40 wt % to about 60 wt % of a gum.

Embodiment 3. A pharmaceutical formulation comprising: (a) about 0.05 wt % to about 2.5 wt % of an opioid; (b) about 45 wt % to about 55 wt % of a polymer selected from the group consisting of a copolymer of polyoxypropylene and polyoxyethylene, and a block copolymer of polyoxypropylene and polyoxyethylene; and (c) about 45 wt % to about 55 wt % of a gum.

Embodiment 4. A pharmaceutical formulation comprising: (a) about 0.1 wt % to about 1.2 wt % of an opioid; (b) about 48 wt % to about 52 wt % of a polymer selected from the group consisting of a copolymer of polyoxypropylene and polyoxyethylene, and a block copolymer of polyoxypropylene and polyoxyethylene; and (c) about 48 wt % to about 52 wt % of a gum.

Embodiment 5. A pharmaceutical formulation comprising: (a) about 0.01 wt % to about 10 wt % of an opioid; (b) about 30 wt % to about 80 wt % of a polymer selected from the group consisting of a copolymer of polyoxypropylene and polyoxyethylene, and a block copolymer of polyoxypropylene and polyoxyethylene; (c) about 20 wt % to about 50 wt % of a gum; and (d) about 0.1 wt % to about 20 wt % of a cellulose compound.

Embodiment 6. A pharmaceutical formulation comprising: (a) about 0.01 wt % to about 5 wt % of an opioid; (b) about 40 wt % to about 75 wt % of a polymer selected from the group consisting of a copolymer of polyoxypropylene and polyoxyethylene, and a block copolymer of polyoxypropylene and polyoxyethylene; (c) about 20 wt % to about 40 wt % of a gum; and (d) about 0.1 wt % to about 20 wt % of a cellulose compound.

Embodiment 7. A pharmaceutical formulation comprising: (a) about 0.05 wt % to about 2.0 wt % of an opioid; (b) about 50 wt % to about 70 wt % of a polymer selected from the group consisting of a copolymer of polyoxypropylene and polyoxyethylene, and a block copolymer of polyoxypropylene and polyoxyethylene; (c) about 25 wt % to about 35 wt % of a gum; and (d) about 1 wt % to about 15 wt % of a cellulose compound.

Embodiment 8. A pharmaceutical formulation comprising: (a) about 0.1 wt % to about 1.2 wt % of an opioid; (b) about 60 wt % to about 65 wt % of a polymer selected from the group consisting of a copolymer of polyoxypropylene and polyoxyethylene, and a block copolymer of polyoxypropylene and polyoxyethylene; (c) about 28 wt % to about 32 wt % of a gum; and (d) about 5 wt % to about 10 wt % of a cellulose compound.

Embodiment 9. The pharmaceutical formulation of any one of Embodiments 1-8, wherein the opioid is an opioid agonist.

Embodiment 10. The pharmaceutical formulation of any one of Embodiments 1-8, wherein the opioid is buprenorphine, adulmine, alfentanil, allocryptopine, allylprodine, alphaprodine, anileridine, aporphine, benzylmorphine, berberine, bicuculine, bicucine, bezitramide, bulbocaprine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tapentadol, tilidine, tramadol, or a pharmaceutically acceptable salt of any of the foregoing.

Embodiment 11. The pharmaceutical formulation of any one of Embodiments 1-8, wherein the opioid is buprenorphine, morphine, hydrocodone, oxycodone, methadone, meperidine, hydromorphone, oxymorphone, fentanyl, or a pharmaceutically acceptable salt of any of the foregoing.

Embodiment 12. The pharmaceutical formulation of any one of Embodiments 1-8, wherein the opioid is buprenorphine free base or a pharmaceutically acceptable salt of buprenorphine.

Embodiment 13. The pharmaceutical formulation of any one of Embodiments 1-8, wherein the opioid is a compound of Formula (I) or a pharmaceutically acceptable salt thereof, a compound of Formula (II) or a pharmaceutically acceptable salt thereof, a compound of Formula (III) or a pharmaceutically acceptable salt thereof, a compound of Formula (IV) or a pharmaceutically acceptable salt thereof, or a compound of Formula (V) or a pharmaceutically acceptable salt thereof.

Embodiment 14. The pharmaceutical formulation of any one of Embodiments 1-8, wherein the opioid is buprenorphine hemiadipate or a pharmaceutically acceptable salt thereof.

Embodiment 15. The pharmaceutical formulation of any one of Embodiments 1-14, wherein the polymer consists of at least one block copolymer of polyoxypropylene and polyoxyethylene.

Embodiment 16. The pharmaceutical formulation of any one of Embodiments 1-15, wherein the polymer is a poloxamer.

Embodiment 17. The pharmaceutical formulation of Embodiment 16, wherein the poloxamer is poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, or a combination of two or more thereof.

Embodiment 18. The pharmaceutical formulation of Embodiment 16, wherein the poloxamer is poloxamer 122, poloxamer 123, poloxamer 124, poloxamer 181, poloxamer 182, poloxamer 183, poloxamer 184, poloxamer 185, poloxamer 188, or a combination of two or more thereof.

Embodiment 19. The pharmaceutical formulation of Embodiment 16, wherein the poloxamer is poloxamer 122, poloxamer 123, poloxamer 124, or a combination of two or more thereof.

Embodiment 20. The pharmaceutical formulation of Embodiment 16, wherein the poloxamer is poloxamer 124.

Embodiment 21. The pharmaceutical formulation of any one of Embodiments 1-20, wherein the gum is guar gum, locust bean gum, tara gum, dextran, carrageenan, xanthan gum, gellan gum, chitan, chitosan, acacia gum, gum arabic, tragacanth gum, karaya gum, mesquite gum, pectin, gum levan, xylan, pullulan, mannan, mannoglucan, carob bean gum, or a combination of two or more thereof.

Embodiment 22. The pharmaceutical formulation of any one of Embodiments 1-20, wherein the gum is carrageenan, dextran, gum arabic, tragacanth gum, pectin, karaya gum, xanthan gum, guar gum, or a combination of two or more thereof.

Embodiment 23. The pharmaceutical formulation of any one of Embodiments 1-20, wherein the gum is xanthan gum, guar gum, or a combination thereof.

Embodiment 24. The pharmaceutical formulation of any one of Embodiments 1-20, wherein the gum is guar gum.

Embodiment 25. The pharmaceutical formulation of any one of Embodiments 5-24, wherein the cellulose compound is an alkyl cellulose, a hydroxyalkyl cellulose, a carboxyalkyl cellulose, or a combination of two or more thereof.

Embodiment 26. The pharmaceutical formulation of any one of Embodiments 5-24, wherein the cellulose compound is methyl cellulose, ethyl cellulose, hydroxypropyl cellulose, hydroxypropylmethyl cellulose, ethylmethyl cellulose, carboxymethyl cellulose, ethylhydroxyethyl cellulose, or a combination of two or more thereof.

Embodiment 27. The pharmaceutical formulation of any one of Embodiments 5-24, wherein the cellulose compound is hydroxypropylmethyl cellulose, carboxymethyl cellulose, or a combination thereof.

Embodiment 28. A method for treating opioid dependence in a human in need thereof comprising administering to the human a therapeutically effective amount of a pharmaceutical formulation of any one of Embodiments 1-27 to treat the opioid dependence.

Embodiment 29. A method for treating pain in a human in need thereof comprising administering to the human a therapeutically effective amount of a pharmaceutical formulation of any one of Embodiments 1-27 to treat the pain.

Embodiment 30. An orally administrable capsule comprising a therapeutically effective amount of a pharmaceutical formulation of any one of Embodiments 1-27.

Embodiment 31. A method for treating opioid dependence in a human in need thereof comprising orally administering to the human the capsule of Embodiment 30 to treat the opioid dependence.

Embodiment 32. A method for treating pain in a human in need thereof comprising orally administering to the human the capsule of Embodiment 30 to treat the pain.

Embodiment 33. An abuse-resistant pharmaceutical formulation comprising: (a) an abuse-susceptible active selected from the group consisting of non-opioid analgesics, non-steroidal anti-inflammatory agents, benzodiazepinres, barbituates, stimulants, and mixtures thereof (b) a nonionic triblock copolymer comprising a central hydrophobic chain of polyoxypropylene flanked by two hydrophilic chains of polyoxyethylene, and having an average molecular weight of 2000 to 2400 Daltons, and (c) a gum, wherein the active agent comprises from 0.1 to 20 wt % of the formulation, the nonionic triblock copolymer comprises from 45 to 70 wt % of the formulation, and the gum comprises from 30 to 50 wt % of the formulation.

Embodiment 34. The abuse resistant formulation of Embodiment 33, wherein the nonionic triblock copolymer comprises from 45 to 65 wt % of the formulation, and the gum comprises from 30 to 50 wt % of the formulation.

Embodiment 35. The abuse resistant formulation of Embodiment 33 or 34, wherein the gum is a guar gum.

Embodiment 36. The abuse resistant formulation of any one of Embodiments 33-35, further comprising an excipient selected from hydroxypropyl methylcellulose, croscarmellose sodium, or mixtures of the two.

Embodiment 37. The abuse resistant formulation of any one of Embodiments 33-36, when extracted into a solvent, selected from water, hot water, and mixtures of ethanol and water, forms a gel that is difficult to filter.

Embodiment 38. A capsule comprising the abuse resistant formulation of any of Embodiments 33-37.

Embodiment 39. The capsule of Embodiment 38 which releases at least 50 wt % of the active within 30 minutes when administered to a buffer solution under the following conditions: USP dissolution apparatus 3, equipped with reciprocating cylinders operating at a stroke rate of 30 min⁻¹ in a pH 2 phosphate buffer.

Embodiment 40. The formulation of any one of Embodiments 1-27 and 33-37 having a viscosity greater than 5000 cp.

Embodiment 41. The capsule of Embodiment 38 which releases at least 85 wt % of the active within 60 minutes when administered to a buffer solution under the following conditions: USP dissolution apparatus 3, equipped with reciprocating cylinders operating at a stroke rate of 30 min⁻¹ in a pH 2 phosphate buffer.

Embodiment 42. The pharmaceutical formulation of any one of Embodiments 1-27, wherein at least 75% of the opioid is released within 10 minutes in a USP Apparatus III dissolution test. The pharmaceutical formulation of any one of Embodiments 1-27, wherein at least 80% of the opioid is released within 10 minutes in a USP Apparatus III dissolution test. The pharmaceutical formulation of any one of Embodiments 1-27, wherein at least 85% of the opioid is released within 10 minutes in a USP Apparatus III dissolution test. The pharmaceutical formulation of any one of Embodiments 1-27, wherein at least 90% of the opioid is released within 10 minutes in a USP Apparatus III dissolution test. The pharmaceutical formulation of any one of Embodiments 1-27, wherein at least 95% of the opioid is released within 10 minutes in a USP Apparatus III dissolution test.

Embodiment 43. The capsule of Embodiment 30, wherein at least 75% of the opioid is released within 10 minutes in a USP Apparatus III dissolution test. The capsule of Embodiment 30, wherein at least 80% of the opioid is released within 10 minutes in a USP Apparatus III dissolution test. The capsule of Embodiment 30, wherein at least 85% of the opioid is released within 10 minutes in a USP Apparatus III dissolution test. The capsule of Embodiment 30, wherein at least 90% of the opioid is released within 10 minutes in a USP Apparatus III dissolution test. The capsule of Embodiment 30, wherein at least 95% of the opioid is released within 10 minutes in a USP Apparatus III dissolution test.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-32. (canceled)

33. An abuse-resistant pharmaceutical formulation comprising,
(a) an abuse-susceptible active selected from the group consisting of non-opioid analgesics, non-steroidal anti-inflammatory agents, benzodiazepinres, barbituates, stimulants, and mixtures thereof,
(b) a nonionic triblock copolymer comprising a central hydrophobic chain of polyoxypropylene flanked by two hydrophilic chains of polyoxyethylene, and having an average molecular weight of 2000 to 2400 Daltons, and
(c) a gum,
wherein the active agent comprises from 0.1 to 20 wt % of the formulation, the nonionic triblock copolymer comprises from 45 to 70 wt % of the formulation, and the gum comprises from 30 to 50 wt % of the formulation.

34. The abuse resistant formulation of claim 33, wherein the nonionic triblock copolymer comprises from 45 to 65 wt % of the formulation, and the guar gum comprises from 30 to 50 wt % of the formulation.

35. The abuse resistant formulation of claim 33, wherein the gum is a guar gum.

36. The abuse resistant formulation of claim 33, further comprising an excipient selected from hydroxypropyl methylcellulose, croscarmellose sodium, or mixtures of the two.

37. The abuse resistant formulation of claim 33, when extracted into a solvent, selected from water, hot water, and mixtures of ethanol and water, forms a gel that is difficult to filter.

38. The abuse resistant formulation of claim 33, wherein said formulation is filled into a capsule.

39. The abuse resistant formulation of claim 38, wherein said capsule releases at least 50 wt % of said active within 30 minutes when administered to a buffer solution under the following conditions: USP dissolution apparatus 3, equipped with reciprocating cylinders operating at a stroke rate of 30 min⁻¹ in a pH 2 phosphate buffer.

40. The abuse resistant formulation of claim 38 having a viscosity of greater than 5000 cp.

41. The abuse resistant formulation of claim 33, wherein the capsule releases at least 85 wt % of the active within 60 minutes when administered to a buffer solution under the following conditions: USP dissolution apparatus 3, equipped with reciprocating cylinders operating at a stroke rate of 30 min⁻¹ in a pH 2 phosphate buffer.

42.-50. (canceled)
